SEQUENCE LISTING (4 sequences)

SEQ ID NO: 1 - DNA, 25 bp, Artificial Sequence
  KDRF1 primer
ggtgacgtgtcctgtattccgttct

SEQ ID NO: 2 - DNA, 31 bp, Artificial Sequence
  KDRF2 primer
gctgactgaaagtaaattggagcgcacaaca

SEQ ID NO: 3 - DNA, 23 bp, Artificial Sequence
  KDRR primer
cgaaattggacaaaagcaaggct

SEQ ID NO: 4 - DNA, 26 bp, Artificial Sequence
  KDR-probe
cgcacaggtacttaaccttttcttag

(19) United States
(12) Patent Application Publication
(10) Pub. No.: US 2012/0115145 A1
(43) Pub. Date: May 10, 2012

(54) STRAND DISPLACEMENT ACTIVITY OF MODIFIED POLYMERASES AND USES THEREOF

(75) Inventor: Guoliang Fu, Oxford (GB)

(73) Assignee: OXITEC LTD, OXFORD (GB)

(21) Appl. No.: 13/289,280

(22) Filed: Nov. 4, 2011

(30) Foreign Application Priority Data
Nov. 5, 2010 (GB) 1018714.4

(51) Int. Cl. C12Q 1/68

(52) U.S. Cl. 435/6.11; 435/6.12

(57) ABSTRACT

The present invention is directed to the use of the strand displacement activity of a modified polymerase. The present invention is more specifically directed to a modified Taq DNA polymerase, which exhibits strand displacement activity, whereas the native polymerase does not possess the strand displacement activity.

### CROSS-REFERENCE TO RELATED APPLICATIONS

The present application claims priority to GB 1018714.4, filed Nov. 5, 2010, which is incorporated by reference herein.

### BACKGROUND

Taq DNA polymerase has been modified extensively. For example, U.S. Pat. No. 5,108,892, describes a nonrecombinant, modified form of thermostable DNA polymerase, which is purified from Thermus aquaticus, having an apparent molecular weight of about 80,000 daltons. The approximate 80,000 dalton molecular weight polymerase exhibits negligible 5′ nuclease activity and is therefore the preferred enzyme for DNA sequencing. The unmodified polymerase can be purified so that the apparent molecular weight by SDS-PAGE is approximately 85,000 daltons. A procedure has been developed for the production of a nonrecombinant, modified form with an apparent molecular weight of about 80,000 daltons. The exact nature of the modification is unknown. However, a proteolytic cleavage event was believed to have occurred. This does not exclude any other protein modifying reactions which alter the molecular weight.

U.S. Pat. No. 5,616,494 describes a recombinant, mutant Taq polymerase, which lacks the N-terminal 235 amino acids of native Taq polymerase. This mutant Taq polymerase has no 5′ nuclease activity.

U.S. Pat. No. 5,436,149 describes a DNA polymerase having an amino acid sequence comprising substantially the same amino acid sequence as that of Thermus aquaticus excluding the N-terminal 280 amino acid residues of Thermus aquaticus DNA polymerase. This mutant Taq polymerase has no 5′ nuclease activity.

U.S. Pat. No. 5,466,591 describes a recombinant thermostable DNA polymerase enzyme which is characterized by the following: (a) in its native form said polymerase comprises a 5′ to 3′ exonuclease domain providing 5′ to 3′ exonuclease activity, wherein said domain comprises an amino acid sequence selected from the group consisting of: A(X)YG wherein X is V or T, (b) said amino acid sequence is mutated in said recombinant enzyme by means other than N-terminal deletion, and (c) said recombinant enzyme has a lesser amount of 5′ to 3′ exonuclease activity than that of the native form of said enzyme.

U.S. Pat. No. 6,130,045 describes a thermostable enzyme having polymerase activity and significantly no nuclease activity. The thermostable enzyme is characterized by comprising a sequence of nine amino acid residues at least proximal to the N-terminus that have less than 50% but greater than 40% amino acid sequence identity with residues 280 to 288 of the naturally occurring Thermus aquaticus polymerase.

The mutant Taq or related DNA polymerases, mentioned above or described in other prior art but not mentioned here, which have mutated 5′ nuclease domain, found applications in sequencing, PCR, primer extension etc.

### BRIEF DESCRIPTION OF THE DRAWINGS

FIG. 1 illustrates the methods using aTaq polymerase for primer extension and strand displacement.

### DETAILED DESCRIPTION

In the present invention, the inventor has surprisingly found that some mutant Taq polymerases exhibit strong strand displacement activity. For example, the aTaq polymerase manufactured by Promega has a strong strand displacement activity.

“Strand displacement activity” designates the phenomenon by which a biological, chemical or physical agent, for example a DNA polymerase, causes the dissociation of a paired nucleic acid from its complementary strand in a direction from 5′ towards 3′, in conjunction with, and close to, the template-dependent nucleic acid synthesis. The strand displacement starts at the 5′ end of a paired nucleic acid sequence and the enzyme therefore carries out the nucleic acid synthesis immediately in 5′ of the displacement site. The neosynthesized nucleic acid and the displaced nucleic acid generally have the same nucleotide sequence, which is complementary to the template nucleic acid strand. The strand displacement activity may be situated on the same molecule as that conferring the activity of nucleic acid synthesis, and particularly the DNA synthesis, or it may be a separate and independent activity. DNA polymerases such as E. coli DNA polymerase I, Klenow fragment of DNA polymerase I, T7 or T5 bacteriophage DNA polymerase, and HIV virus reverse transcriptase are enzymes which possess both the polymerase activity and the strand displacement activity. Agents such as helicases can be used in conjunction with inducing agents which do not possess strand displacement activity in order to produce the strand displacement effect, that is to say the displacement of a nucleic acid coupled to the synthesis of a nucleic acid of the same sequence. Likewise, proteins such as Rec A or Single Strand Binding Protein from E. coli or from another organism could be used to produce or to promote the strand displacement, in conjunction with other inducing agents (KORNBERG, A. and BAKER T. A. 1992, DNA Replication, 2nd Edition, pp 113-225, Freeman, N.Y.).

Strand displacement activity is a well known property of certain DNA polymerases (Sambrook et al., 1989. Molecular Cloning: A Laboratory Manual, 2nd Edition, pp. 5.33-5.35, Cold Spring Harbor Laboratory, Cold Spring Harbor). The properties of DNA polymerases, especially the strand displacement activity of some of them are detailed by Kornberg and Baker (1992, DNA Replication, 2nd Edition, pp. 113-225, Freeman, N.Y.). The strand displacement activity was initially demonstrated for the Klenow fragment of DNA polymerase I of Escherichia coli (Masamune and Richardson, 1971. J. Biol. Chem. 246: 2692-2701), which confers on this enzyme the capacity to initiate the replication of a nucleic acid from the 3′ OH end of a break in a double-stranded DNA. This strand displacement property is limited in the case where the DNA polymerases possess a 5′-3′ exonuclease activity (Lundquist and Olivera, 1982. Cell 31 : 53-60). This strand displacement activity has also been demonstrated in thermostable DNA polymerases such as Tli DNA polymerase (Kong et al., 1993. J. Biol. Chem. 268 : 1965-1975). In this case, it was also shown that the mutated forms of this enzyme, not having 5′-3′ exonuclease activity, have a greater strand displacement capacity. Strand displacement is not a property common to all DNA polymerases since some of them, like T4 DNA polymerases, Taq DNA polymerase, are not capable of carrying out, on their own, strand displacement. This strand displacement activity has also been demonstrated for T7 DNA polymerase (Lechner et al., 1983. J. Biol. Chem. 258 : 11174-11184) and for HIV reverse transcriptase (Huber et al., 1989. J. Biol. Chem. 264 : 4669-4678). DNA polymerases having a strand displacement capacity, and more particularly being capable of initiating polymerization (from 5′ towards 3′) from the 3′ OH end of a break in a double-stranded DNA is useful for carrying out the amplification reaction of the present invention. Preferably, a DNA polymerase lacking 5′-3′ exonuclease activity is used for carrying out the amplification cycle, since the efficiency of the strand displacement activity is greater in enzymes lacking this activity. The Klenow fragment of DNA polymerase I of Escherichia coli constitutes an example of a polymerase lacking 5′-3′ exonuclease activity, likewise polymerases such as T4 DNA polymerase, T7 DNA polymerase or Sequenase (US Biochemical), T5 DNA polymerase or Phi29 DNA polymerase could also be used. However, the present invention also comprises the use of DNA polymerases having this 5′-3′ exonuclease activity when the latter does not prevent the implementation of the amplification method. In this case, the yield of the amplification reaction can be enhanced by specific inhibition of the 5′-3′ exonuclease activity of DNA polymerases under the reaction conditions used.

The present invention provides a method for assaying a sample for one or more target nucleic acids in a reaction using a modified polymerase, said method comprises primer extension and strand displacement, wherein said modified polymerase is modified from a native polymerase such that the modified form exhibits the strand displacement activity, whereas the native, unmodified polymerase does not possess the strand displacement activity. It is preferred that said modified polymerase is a modified form of DNA polymerase derived from thermophilic bacteria that belong to the Deinococcus-Thermus group, wherein the modified form of DNA polymerase lacks 5′ nuclease activity, but gains new strand displacement activity. It is preferred that the modified form of DNA polymerase is derived from Thermus aquaticus.

In one aspect, the modified form of DNA polymerase is a nonrecombinant, modified form of DNA polymerase derived from Thermus genus. For example, the nonrecombinant, modified form of Taq DNA polymerase is aTaq DNA polymerase manufactured by Promega. The nonrecombinant, modified form of Taq DNA polymerase may be prepared by a process described in U.S. Pat. No. 5,108,892 or by a modified form of said process.

As “892” patent described, it was believed that the polymerase modification involves cleavage by one or more proteases to inactivate a 5′-3′ exonuclease. The modification clearly alters the apparent molecular weight of the polymerase, and the degree of molecular weight shift to the modified form varies inversely with the exonuclease activity, as seen in DNA sequencing reactions. Thus, the amount of exonuclease activity is inversely proportional to the amount of 80,000 dalton molecular weight modified Taq DNA polymerase that is present. The more 80,000 dalton molecular weight protein that is present, the less exonuclease activity is seen. The nature of the modification may not be enzyme mediated proteolysis, but an alternative modification, such as protein oxidation, which would alter the apparent molecular weight.

Although the “892” patent described many uses for the modified Taq polymerase, the strong strand displacement activity had not been discovered and was not used.

In another aspect of the invention, the modified form of DNA polymerase may be a recombinant, modified form of DNA polymerase from Thermus genus. Preferably, the enzyme is a modified form of Taq polymerase. The modified form of DNA polymerase may have the same length as the native enzyme, but with a mutated 5′ nuclease domain. An example of one such mutant enzyme may be the one described in U.S. Pat. No. 5,466,591.

The modified form of DNA polymerase may be a truncated form of the native enzyme with up to 50 amino acids deleted from the N-terminal of the native enzyme. The modified form of DNA polymerase may be a truncated form of the native enzyme with up to 100 amino acids deleted from the N-terminal of the native enzyme. Preferably, the modified form of DNA polymerase may be a truncated form of the native enzyme with up to 235 amino acids deleted from the N-terminal of the native enzyme. An example of one such mutant enzyme may be the one described in U.S. Pat. No. 5,616,494.

The modified form of DNA polymerase may also be a truncated form of the native enzyme with up to 280 amino acids deleted from the N-terminal of the native enzyme. An example of one such mutant enzyme may be the one described in U.S. Pat. No. 5,436,149.

In a further aspect, the modified form of DNA polymerase may be a fusion protein with an amino acid fragment fused with the N-terminus or/and C-terminus of a mutated polymerase which lacks 5′ nuclease activity, wherein said fused amino acid fragment confers the strand displacement activity on the enzyme. The amino acid fragment may be chosen from a group of peptide fragments which exhibit strand displacement activity or helicase activity, such amino acid fragments may be derived from helicase, Rec A or Single Strand Binding Protein and other amino sequences, which confer strand displacement activity of the fused enzyme.

The U.S. Pub. No. 2006/0234227 describes a fusion enzyme of helix-harpin-helix motifs with DNA polymerase. Our experiment showed that this fusion enzyme does not exhibit the strand displacement activity.

The strand displacement activity discovered in the modified DNA polymerase in the present invention can be very useful in a reaction for detection or quantification of a target nucleic acid.

The present invention provides methods for assaying a sample for one or more target nucleic acids in a reaction using modified polymerase, said method comprises primer extension and strand displacement, wherein said modified polymerase is modified from a native polymerase such that the modified form exhibits the strand displacement activity, whereas the native, unmodified polymerase does not possess the strand displacement activity, wherein said modified polymerase is a modified form of DNA polymerase derived from Thermus genus, for example Taq DNA polymerase from Thermus aquaticus, wherein said modified form of DNA polymerase lacks 5′ nuclease activity,

wherein said modified form of Taq DNA polymerase may be a non-recombinant, modified form of Taq DNA polymerase,

wherein said non-recombinant, modified form of Taq DNA polymerase may be aTaq DNA polymerase manufactured by Promega,

wherein said non-recombinant, modified form of Taq DNA polymerase may be prepared by a process described in U.S. Pat. No. 5,108,892 or by a modified form of said process.

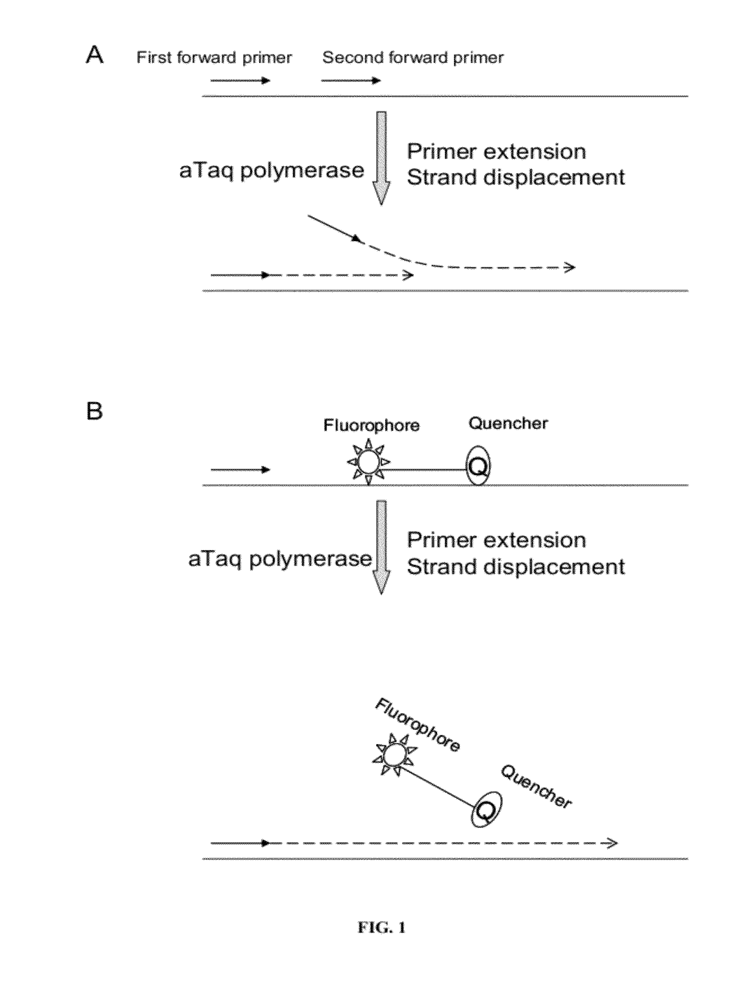

In one embodiment the reaction may comprise at least two forward primers: first forward primer and second forward primer, capable of annealing to the same strand of a target sequence, wherein the first primer anneals the target sequence upstream of the second primer, wherein under extension conditions the modified Taq polymerase extends both primers at the same time, the first primer extension strand displaces the second primer extension strand (FIG. 1A).

In another embodiment, the reaction comprises a primer and a labeled probe, wherein the primer and the labeled probe hybridise to the same strand of a target sequence, the primer anneals the target sequence upstream of the labeled probe, wherein under extension conditions the modified polymerase extends the primers, at the same time the primer extension strand displaces the labeled probe (FIG. 1B). The labeled probe may be long and may have a high Tm (melting temperature). The Tm of the labeled probe of the present invention can be higher than the temperature used in the extension conditions. Traditional DNA polymerase can not pass through the labeled probe annealing upstream of the primer with a Tm above the extension temperature. However, the modified Taq polymerase (aTaq polymerase from Promega) can effectively displace the labeled probe annealing upstream of the primer with a Tm above the extension temperature, and extend the primer without any difficulty.

The labeled probe comprises a reporter label and a quencher label. The later is capable of quenching the fluorescence of said reporter label when said oligonucleotide probe is in a single-stranded conformation and is not hybridized to said target nucleic acid. The probe is capable of forming a double stranded conformation when hybridized to said target nucleic acid, where the fluorescence of said reporter label is unquenched such that the fluorescence intensity of said reporter label is greater than the fluorescence intensity of said reporter label when said oligonucleotide probe is in a single stranded conformation and not hybridized to said target nucleic acid.

The length of the probe, not hydrolysable, is not limited, and can be designed as long as one wishes. In the present invention, a long probe with a high Tm can be used. The Tm of the probe can be designed higher than the elongation temperature such as 72° C. or above. The quencher label is preferably a non-fluorescent dye label, which is attached to the 3′ terminus or 5′ terminus of the oligonucleotide probe, or to an internal residue of the oligonucleotide probe. The reporter label is a fluorescent dye label, which is attached to an internal residue of the oligonucleotide probe, or is attached to the 5′ terminus or 3′ terminus of the oligonucleotide probe. It is preferred that the probe is labeled with a fluorophore at the 5′ terminus and a quencher at the 3′ terminus. Alternatively, the probe is labeled with a fluorophore at an internal nucleotide and with a quencher at the 3′ terminus. The reporter label is preferably a fluorophore, which may be selected from the group consisting of fluorescein, fluorescein derivatives, cyanine dyes, fluorescein-cyanine conjugates, and a similar.

As used herein, the term “quencher” includes any moiety that is capable of absorbing the energy of an excited fluorescent label, when it is located in close proximity to the fluorescent label and is capable of dissipating that energy. A quencher can be a fluorescent quencher or a non-fluorescent quencher, the later is also referred to as a dark quencher. The fluorophores listed above can play a quencher role if brought into proximity to another fluorophore, wherein either FRET quenching or contact quenching can occur. It is preferred that a dark quencher which does not emit any visible light is used.

In a further embodiment, the reaction may comprise at least two forward primers: first forward primer and second forward primer, and a labeled probe capable of annealing to the same strand of a target sequence, wherein the first primer anneals the target sequence upstream of the second primer, and the second primer anneals the target sequence upstream of the labeled probe, wherein under extension conditions the modified Taq polymerase extends both primers at the same time, the first primer extension strand displaces the second primer extension strand, and the second primer extension strand displaces the labeled probe.

The first primer and the second primer anneal to the target nucleic acid such that the 3′ end of the first primer is adjacent to or upstream of the 5′ end of the second primer. When two different, non-overlapping oligonucleotides anneal to different regions of the same linear, complementary nucleic acid sequence, and the 3′ end of one oligonucleotide points toward the 5′ end of the other, the former may be called the “upstream” oligonucleotide and the latter the “downstream” oligonucleotide. Herein, the first primer is an upstream primer.

For the present invention used in amplification reaction, there may be one forward primer, or preferably two or more forward primers; there may be one reverse primer, or preferably two or more reverse primers

According to the present invention, the reaction may be a PCR (polymerase chain reaction) or PCDR (polymerase chain displacement reaction, PCT/GB07/03793) amplification, which comprises normal PCR thermal cycling conditions, including cycles containing a denaturing step, an annealing step and an extension step, or it is two step PCR containing a denaturing step and a combined annealing-extension step.

It is preferred that when the reaction is a PCR or PCDR amplification, the PCR comprises modified PCR thermal cycling conditions, wherein in each cycle, the thermal cycling program comprises one denaturing step and at least two rounds of an annealing step and/or extension step. For example, in each cycle there is one denaturing step, first annealing step, first extension step, second annealing step and second extension step. Alternatively, in each cycle there is one denaturing step, first annealing step, first extension step and a combined second annealing-extension step (ie one temperature). In each cycle there could also be one denaturing step, first annealing step, first extension step and second annealing step without a second extension step. The first and second annealing steps may be the same or different temperatures, and the first and second extension steps may be the same or different temperatures.

In a further embodiment, when the reaction is a PCR or PCDR amplification, and comprises a labeled probe, the method may comprise a melting curve analysis at the end of amplification. The labeled probe may be designed to hybridise to a variant region of the target sequence, wherein the melting curve profile indicates whether or not a particular sequence is present.

It is preferred that when the reaction is a PCR or PCDR amplification, and comprises a labeled probe, the PCR amplification is an asymmetric reaction with an unequal mole ratio of forward and reverse primers. When the forward primer and labeled probe hybridise to the same strand of a target sequence, the ratio of reverse/forward primers should be more than one, preferably more than 1.5. Alternatively, the concentration of the reverse primer is at least two-fold higher than the concentration of any of the forward primers.

The modified form of Taq DNA polymerase may be a recombinant, modified form of Taq DNA polymerase which lacks 5′ nuclease activity, but possessing strand displacement activity. The modified form of Taq DNA polymerase may have the same length as the native enzyme, but with a mutated 5′ nuclease domain, or it may be a truncated form of the native enzyme with 50-280 amino acids deleted from the N-terminal of the native enzyme.

The modified form of Taq DNA polymerase may be a fusion protein with an amino acid fragment fused with the N-terminus of a mutated Taq polymerase which lacks 5′ nuclease activity, wherein said amino acid fragment confers the strand displacement activity on the enzyme.

The reaction in the present invention may be an isothermal amplification reaction, which may be SDA, NASBA, RCA, LAMP, ICAN, TCA, or MDA. The reaction may be a whole genome amplification reaction, which utilises primers with wobble nucleotides.

It is preferred that the reaction comprises betaine.

The present invention also provides a method for assaying a sample for one or more target nucleic acids in a reaction using asymmetric amplification, said method comprises primer extension and strand displacement, wherein said reaction comprises at least two forward primers: first forward primer and second forward primer, capable of annealing to the same strand of a target sequence, and one or more reverse primers, wherein the first forward primer anneals the target sequence upstream of the second forward primer, wherein under extension conditions a DNA polymerase (or modified DNA polymerase) with strand displacement activity extends both primers at the same time, the first primer extension strand displaces the second primer extension strand, wherein the concentration of the reverse primer is at least 1.5-fold or preferably at least two-fold higher than the concentration of any of the forward primers.

There are many applications where asymmetric amplification is desirable, for example, the amplification product is identified by probing with a labelled oligonucleotide. Currently, this is mainly achieved by using more of one primer than of the other. Ideally, exponential amplification of the target sequence takes place until one primer is exhausted and linear amplification then results. Due to the unequal primer concentration affecting the early stages of the reaction, there is often a problem in that the PCR process does not start properly. Therefore, the present invention includes a method of conducting an asymmetric nucleic acid amplification involving multiple primer extension and strand displacement. The traditional asymmetric PCR uses a single primer, for example a forward primer, which has a low concentration (rate limited). If this forward primer does not start the reaction properly, the whole amplification may fail. The current method uses at least two forward primers, which have a lower concentration than the reverse primer. The two forward primers are extended by a DNA polymerase with strand displacement activity simultaneously under extension conditions; the upstream forward primer extension product displaces the downstream forward primer extension product. As there is more than one forward primer, although at a low concentration, the combined effect of the forward primers results in the efficient start of the amplification reaction. At a later stage of PCR amplification, the forward primers with low concentration are running out, linear amplification occurs, and this results in single-stranded end products. In this method, any DNA polymerase with strand displacement activity can be used. Thermostable DNA polymerase is preferred. Modified Thermus genus derived polymerase with displacement activity, as disclosed in this invention, is most preferable. The ratio between the reverse primer and any of the low-concentration forward primers can be more than one, preferably more than 1.5, but most preferable is more than 2. Generally, amplification includes multiple primers (at least two primers) in low concentration for one side (forward side) of PCR. However, the high-concentration primer side (reverse side) can also comprise multiple primers. For some applications, it may be desirable for both forward and reverse sides to comprise multiple primers. In this case, for asymmetric amplification to occur, the concentration of the total reverse primers (if there are multiple reverse primers) is higher than the concentration of the total forward primers (if there are multiple forward primers). The ratio of concentration between the total reverse primers and the total forward primers is at least 1.5, or at least 2, or at least 3 or at least 4 or at least 5. Alternatively, the concentration of one of the reverse primers with the highest concentration should be at least 1.5-fold or preferably at least two-fold higher than the concentration of any of the multiple forward primers. A higher concentration ratio, such as 3, 4, 5, 6, 7, 8, 9, or 10-fold between the reverse primer with the highest concentration among the multiple reverse primers and any of the multiple forward primers may be also used or may be preferred.

In another embodiment of the present invention, the modified DNA polymerase is made hot-start. Both antibody and chemical modification can make the DNA polymerase of the present invention hot-start.

The present invention further provides a kit for assaying for one or more nucleic acid targets in a reaction using a modified polymerase, which comprises a modified Taq polymerase having strand displacement activity according to any one of the preceding claims comprising:

at least two forward primers: first primer and second primer, capable of annealing to the same strand of a target sequence, wherein the first primer anneals the target sequence upstream of the second primer, wherein under extension conditions the modified Taq polymerase extends both primers at the same time, the first primer extension strand displaces the second primer extension strand,

at least one reverse primer,

or a primer and a labeled probe, wherein the primer and the labeled probe hybridise to the same strand of a target sequence, the primer anneals the target sequence upstream of the labeled probe, wherein under extension conditions the modified Taq polymerase extends the primers, at the same time the primer extension strand displaces the labeled probe.

In the kit, for asymmetric amplification, the concentration of the reverse primer is at least 1.5 or preferably two fold higher than the concentration of any of the forward primers.

### EXAMPLE 1

All primers used in the subsequent experiments were synthesized by EUROGENTEC. Primers were designed to amplify a target DNA sequence voltage-gated sodium gene, a mutation of which is associated with pyrethroid resistance in Latin American Aedes aegypti. Mutation at codon Val1016 changing to iso1016 or Gly1016 confer KDA (knockdown resistance to pyrethroids). The sequences of the primers are:

(SEQ ID NO: 1)
KDRF1: GGTGACGTGTCCTGTATTCCGTTCT
(SEQ ID NO: 2)
KDRF2: GCTGACTGAAAGTAAATTGGAGCGCACAACA
(SEQ ID NO: 3)
KDRR: CGAAATTGGACAAAAGCAAGGCT

Probe targeting to Val1016 region is designed as follows:

KDR-probe: 5′ Fam-CGCACAGGTACTTAACCTTTTCTTAG-BHQ1 3′ (SEQ ID NO: 4), which is labeled with Fam at the 5′ end and BHQ1 at the 3′ end. All primers and probe were diluted to 10 μM, as working solutions.

All nucleic acid sequences are written 5′ to 3′ unless otherwise stated. Genotyping for KDR resistance was carried out using a Stratagene MX3005P Real Time PCR machine.

Perform amplification using the following ingredients and conditions: 10×PCR Buffer (Thermophilic DNA Polymerase 10× Reaction Buffer, Mg-Free) 2 μl, MgCl₂ (Magnesium Chloride Solution, 25 mM) 1.8 μl, 10 mM dNTPs 0.4 μl, forward primer KDRF1 0.2 μl, forward primer KDRF2 0.08 μl, reverse primer KDRR 0.6 μl, aTaq DNA polymerase (Promega 5 u/μl) 0.08 μl, Probe 0.6 μl, genomic DNA 2 μl (Extracted using Fermentas gDNA extraction kit) and water to final volume of 20 μl.

This is an asymmetric PCR. The concentration of the reverse primer, which is 300 nM, is greater than the individual forward primer concentration which is 100 nM for KDRF1 and 40 nM for KDRF2.

Reactions were carried out at 95° C. 60 sec; 45 cycles of 95° C. 6 sec, 55° C. 20 sec, 72° C. 15 sec, 57° C. 15 sec, 72° C. 15 sec. Fluorescence measurements were recorded during the read steps at 55° C. and 57° C. Post-amplification melting profile had the following conditions: after the last cycle of PCR, heat at 95° C. for 10 sec, cool to 50° C. and hold for 30 sec, then slowly increase the temperature to 74° C. The fluorescence emission data is continually collected during the rising temperatures. The first negative derivative of the emission reading, with respect to temperature, is plotted against the temperature to form the melting curves.

Other experiments were carried out in a reaction which contained, apart from the same ingredients as above, 1M or 1.2M betaine. Adding betaine could help strand-displacement, amplify difficult templates, reduce non-specific amplification, or reduce primer-dimer.

1. A method for assaying a sample for one or more target nucleic acids in a reaction using modified polymerase, comprising primer extension and strand displacement, wherein said modified polymerase is modified from a native polymerase such that the modified form exhibits the strand displacement activity, whereas the native, unmodified polymerase does not possess the strand displacement activity, wherein said modified polymerase is a modified form of DNA polymerase from thermophilic bacteria that belong to the Deinococcus-Thermus group, wherein said modified form of DNA polymerase lacks 5′ nuclease activity, but gains new strand displacement activity.

2. The method according to claim 1, wherein said modified form of DNA polymerase is derived from Thermus aquaticus.

3. The method according to claim 2, wherein said modified form of DNA polymerase is a modified form of Taq DNA polymerase.

4. The method according to claim 3, wherein said modified form of Taq DNA polymerase is nonrecombinant.

5. The method according to claim 4, wherein said nonrecombinant, modified form of Taq DNA polymerase is prepared by a process described in U.S. Pat. No. 5,108,892 or by a modified form of said process.

6. The method according to claim 1, wherein the reaction comprises at least two forward primers: first forward primer and second forward primer, capable of annealing to the same strand of a target sequence, wherein the first primer anneals the target sequence upstream of the second primer, wherein under extension conditions the modified form of DNA polymerase extends both primers, the first primer extension strand displaces the second primer extension strand.

7. The method according to claim 1, wherein said reaction is a PCR (polymerase chain reaction) or PCDR (polymerase chain displacement reaction) amplification, which comprises normal PCR thermal cycling conditions such as a denaturing step, annealing step and extension step, or contains a denaturing step and combined annealing-extension step.

8. The method according to claim 1, wherein said reaction is a PCR or PCDR amplification, which comprises modified PCR thermal cycling conditions, wherein in each cycle the thermal cycling program comprises one denaturing step and at least two rounds of annealing step and/or extension step, for example, in each cycle there is a denaturing step, first annealing step, first extension step, second annealing step, and an optional second extension step, wherein the first and second annealing steps are the same or different temperatures, and the first and second extension steps are the same or different temperatures.

9. The method according to claim 1, wherein said reaction is a PCR or PCDR amplification, comprising a labeled probe, and the method comprises a melting curve analysis of the probe hybridising to the target sequence at the end of amplification wherein said PCR or PCDR amplification is an asymmetric amplification with unequal mole ratio of forward and reverse primer.

10. The method according to claim 1, wherein said modified form of DNA polymerase is a recombinant, modified form of Taq DNA polymerase which lacks 5′ nuclease activity, but possesses strand displacement activity.

11. The method according to claim 10, wherein said modified form of Taq DNA polymerase has the same length as the native enzyme, but it has a mutated 5′ nuclease domain, or it is a truncated form of the native enzyme with up to 50-280 amino acids deleted from the N-terminal of the native enzyme.

12. The method according to claim 10, wherein said modified form of Taq DNA polymerase is a fusion protein with an amino acid fragment fused with the N-terminus or C-terminus of a mutated Taq polymerase which lacks 5′ nuclease activity, wherein said fused amino acid fragment confers the strand displacement activity on the enzyme.

13. The method according to claim 1, wherein said reaction is an isothermal amplification reaction.

14. The method according to claim 1, wherein said reaction is a whole genome amplification reaction, which utilises primers with wobble nucleotides.

15. The method according to claim 1, wherein the reaction comprises betaine.

16. The method according to claim 9, wherein said probe comprises a reporter label and a quencher label, wherein the quencher label is capable of quenching the fluorescence of said reporter label when said oligonucleotide probe is in a single-stranded conformation and is not hybridized to said target nucleic acid,
wherein said oligonucleotide probe is capable of forming a double stranded conformation when hybridized to said target nucleic acid, where the fluorescence of said reporter label is unquenched such that the fluorescence intensity of said reporter label is greater than the fluorescence intensity of said reporter label when said oligonucleotide probe is in a single stranded conformation not hybridized to said target nucleic acid.

17. A method for assaying a sample for one or more target nucleic acids in a reaction using asymmetric amplification, wherein said method comprises primer extension and strand displacement, and said reaction comprises at least two forward primers: first forward primer and second forward primer, capable of annealing to the same strand of a target sequence, and at least one reverse primer, wherein the first forward primer anneals the target sequence upstream of the second forward primer, wherein under extension conditions a modified DNA polymerase with strand displacement activity extends both primers, the first primer extension strand displaces the second primer extension strand, wherein the concentration of the total reverse primers is at least two-fold higher than the concentration of the total forward primers.

18. A kit for assaying for one or more nucleic acid targets in a reaction using modified polymerase, which comprises a modified Taq polymerase with strand displacement activity according to any one of the preceding claims comprising:
at least two forward primers: first primer and second primer, capable of annealing to the same strand of a target sequence, wherein the first primer anneals the target sequence upstream of the second primer, wherein under extension conditions the modified Taq polymerase extends both primers, the first primer extension strand displaces the second primer extension strand,
at least one reverse primer,
or a primer and a labeled probe, wherein the primer and the labeled probe hybridise to the same strand of a target sequence, the primer anneals the target sequence upstream of the labeled probe, wherein under extension conditions the modified Taq polymerase extends the primer, at the same time the primer extension strand displaces the labeled probe,
wherein the concentration of reverse primer is at least two-fold higher than the concentration of any of the forward primers.
